(19) United States
(12) Patent Application Publication
(10) Pub. No.: US 2003/0147968 A1
(43) Pub. Date: Aug. 7, 2003

(54) Allantoin-containing preparations for administration as gels and aerosols

(75) Inventor: Elliott Farber, North Mankato, MN (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Alwyn Company, Inc.

(21) Appl. No.: 10/322,797

(22) Filed: Dec. 19, 2002

Related U.S. Application Data
(60) Provisional application No. 60/341,906, filed on Dec. 19, 2001.
(60) Provisional application No. 60/349,147, filed on Jan. 15, 2002.

(51) Int. Cl. A61K 31/4162; A61K 35/60

(52) U.S. Cl. 424/523; 514/386

(57) ABSTRACT

Allantoin-containing preparations are prepared in a form suitable for administration as gels and aerosols. In one embodiment of the aerosol formulation, an allantoin-containing composition comprises: (1) water; (2) allantoin in a therapeutically effective quantity; (3) an anionic ethoxylated surfactant; (4) lanolin oil; and (5) cod liver oil. The composition has a viscosity and surface tension in a range that allows the composition to be administered to the skin of a user as an aerosol by a mechanical spray pump. In another aspect of this invention, the allantoin-containing preparations are prepared in a form suitable for administration as gels. The compositions can be substantially anhydrous or can contain water. In one embodiment of the gel formation, an allantoin-containing composition comprises: (1) allantoin in a therapeutically effective quantity; (2) a complex comprising mineral oil and at least one hydrogenated alkylene copolymer; (3) lanolin oil; and (4) cod liver oil. The compositions can comprise other ingredients. Compositions according to the present invention are suitable for the treatment of a number of skin conditions or diseases characterized by ulceration, inflammation, or blistering of the skin, such as epidermolysis bullosa.

### CROSS-REFERENCE TO RELATED APPLICATIONS

This application claims the priority from U.S. Provisional Patent Application No. 60/341,906, filed Dec. 19, 2001, entitled “Allantoin-Containing Preparations for Administration as Gels,” and from U.S. Provisional Patent Application No. 60/349,147, filed Jan. 15, 2002, entitled “Allantoin-Containing Preparations for Administration as Aerosols.”

### BACKGROUND OF THE INVENTION

1. Field of the Invention

This invention is directed to allantoin-containing preparations suitable for administration in gel form and as aerosols.

2. Description of Related Art

Allantoin is a commonly used ingredient in cosmetic applications, where it exerts a skin-protective function. There is a particular need for an easy-to-use allantoin composition that can be administered to patients suffering from a variety of skin diseases and conditions.

In particular, there is a need for compositions that are suitable for treating a number of severe and difficult-to-treat skin conditions that are characterized by ulceration, inflammation, and/or blistering of the skin. One of these skin conditions is epidermolysis bullosa. This is a severe genetic skin disorder in which the skin breaks down and large blisters appear. These blisters are difficult to treat by conventional means. Other skin diseases for which improved treatments are needed are pressure ulcers, decubitus ulcers or bedsores, diabetic ulcers, and milia, as well as other conditions affecting the skin and having an inflammatory component such as burns, eczema, urticaria, atopic dermatitis, contact dermatitis, arthritis, gout, and lupus erythematosus. Therefore, improved compositions that are suitable for treating these diseases are needed.

### SUMMARY OF THE INVENTION

One aspect of the present invention is an allantoin-containing composition in gel form. The composition can be substantially anhydrous or can contain water.

One embodiment of a composition according to this aspect of the present invention comprises:

(1) allantoin in a therapeutically effective quantity;

(2) a complex comprising mineral oil and at least one hydrogenated alkylene copolymer;

(3) lanolin oil; and

(4) cod liver oil.

Typically, at least one monomer in the hydrogenated alkylene copolymer is styrene. Preferably, the hydrogenated alkylene copolymer includes both a hydrogenated butylene/ethylene/styrene copolymer and a hydrogenated ethylene/propylene/styrene copolymer.

The composition can further comprise other ingredients, such as a solvent component, an antioxidant component, a preservative component, and fragrance.

A preferred composition of this embodiment of the invention comprises:

(1) allantoin in a therapeutically effective quantity;

(2) a complex comprising:

(a) mineral oil;

(b) a hydrogenated butylene/ethylene/styrene copolymer; and

(c) a hydrogenated ethylene/propylene/styrene copolymer;

(3) lanolin;

(4) cod liver oil;

(5) butylated hydroxytoluene;

(6) propylparaben;

(7) fragrance; and

(8) propylene glycol.

Another embodiment of a composition according to this aspect of the present invention comprises:

(1) allantoin in a therapeutically effective quantity;

(2) water;

(3) a complex comprising mineral oil and at least one hydrogenated alkylene copolymer;

(4) lanolin oil; and

(5) cod liver oil.

The complex comprising mineral oil and at least one hydrogenated alkylene copolymer is as described above.

This embodiment of the composition can comprise other ingredients, such as a solvent component, an antioxidant component, a preservative component, and fragrance.

A preferred composition of this embodiment of the invention comprises:

(1) allantoin in an therapeutically effective quantity;

(2) water;

(3) a complex comprising:

(a) mineral oil;

(b) a hydrogenated butylene/ethylene/styrene copolymer; and

(c) a hydrogenated ethylene/propylene/styrene copolymer;

(4) lanolin oil;

(5) cod liver oil;

(6) butylated hydroxytoluene;

(7) propylparaben;

(8) fragrance; and

(9) propylene glycol.

Yet another embodiment of a composition according to the present invention comprises:

(1) allantoin in a therapeutically effective quantity;

(2) a polymer selected from the group consisting of polyglyceryl methacrylate, polyglyceryl acrylate, and polyglyceryl ethacrylate;

(3) lanolin oil; and

(4) cod liver oil.

Preferably, the polymer is polyglyceryl methacrylate.

This embodiment of the composition can further comprise other ingredients, such as a solvent component, an antioxidant component, a preservative component, and fragrance.

A preferred composition of this embodiment of the invention comprises:

(1) allantoin in a therapeutically effective quantity;

(2) polyglyceryl methacrylate;

(3) lanolin oil;

(4) cod liver oil;

(5) butylated hydroxytoluene;

(6) propylparaben;

(7) propylene glycol; and

(8) fragrance.

Yet another embodiment of a composition according to the present invention comprises:

(1) allantoin in a therapeutically effective quantity;

(2) water;

(3) a polymer selected from the group consisting of polyglyceryl methacrylate, polyglyceryl acrylate, and polyglyceryl ethacrylate;

(4) lanolin oil; and

(5) cod liver oil.

Preferably, the polymer is polyglyceryl methacrylate.

This embodiment of the composition can further comprise other ingredients, such as a solvent component, an antioxidant component, a preservative component, and fragrance.

A preferred composition of this embodiment of the present invention comprises:

(1) allantoin in a therapeutically effective quantity;

(2) water;

(3) polyglyceryl methacrylate;

(4) lanolin oil;

(5) cod liver oil;

(6) butylated hydroxytoluene;

(7) propylparaben;

(8) fragrance; and

(9) propylene glycol.

Yet another embodiment of a composition according to the present invention comprises:

(1) water;

(2) allantoin in a therapeutically effective quantity;

(3) a carboxypolymethylene polymer;

(4) PEG-100 stearate;

(5) lanolin oil;

(6) cod liver oil; and

(7) a water-soluble base to adjust the pH from about 3.5-6.0.

Typically, the water-soluble base is sodium hydroxide. Preferably, the water-soluble base adjusts the pH to about 4.8. Preferably, the carboxypolymethylene polymer is a cross-linked polyacrylic acid that is cross-linked by allylsucrose or allylpentaerythritol with the polyacrylic acid.

This embodiment of the composition can further comprise other ingredients, such as a solvent component, an antioxidant component, and a preservative component.

A preferred composition of this embodiment of the present invention comprises:

(1) water;

(2) allantoin in a therapeutically effective quantity;

(3) a cross-linked polyacrylic acid that is cross-linked by allylsucrose or allylpentaerythritol with the polyacrylic acid;

(4) PEG-100 stearate;

(5) lanolin oil;

(6) cod liver oil;

(7) sodium hydroxide;

(8) propylene glycol;

(9) butylated hydroxytoluene; and

(10) 2-methyl-4-isothiazolin-3-one.

Yet another embodiment of a composition according to the present invention comprises:

(1) water;

(2) allantoin in a therapeutically effective quantity;

(3) a carboxypolymethylene polymer;

(4) lanolin oil;

(5) cod liver oil;

(6) a complex comprising:

(a) a polymer selected from the group consisting of glycerylpolyacrylate, glycerylpolymethacrylate, and glycerylpolyethacrylate; and

(b) glycerin; and

(7) a weak organic base to adjust the pH.

The carboxypolymethylene polymer is as described above.

Preferably, the polymer in the complex is glycerylpolyacrylate.

Preferably, the weak organic base is triethanolamine.

This embodiment of the composition can further comprise other ingredients, such as a solvent component, an antioxidant component, a preservative component, sodium lauroamphoacetate, and fragrance.

A preferred composition of this embodiment of the invention comprises:

(1) water;

(2) allantoin in a therapeutically effective quantity;

(3) a cross-linked polyacrylic acid that is cross-linked by allylsucrose or allylpentaerythritol with a polyacrylic acid;

(4) lanolin oil;

(5) cod liver oil;

(6) a complex comprising glycerylpolyacrylate and glycerin;

(7) triethanolamine;

(8) propylene glycol;

(9) sodium lauroamphoacetate;

(10) butylated hydroxytoluene;

(11) fragrance;

(12) propylparaben; and

(13) methylparaben.

Another aspect of the present invention is allantoin-containing compositions that are suitable for administration as aerosols.

One embodiment of an allantoin-containing composition according to this aspect of the present invention comprises:

(1) water;

(2) allantoin in a therapeutically effective quantity;

(3) an anionic ethoxylated surfactant;

(4) lanolin oil;

(5) a substantially anhydrous hydroxylic solvent in a quantity sufficient to dissolve the allantoin and reduce the surface tension so that aerosol droplets can be formed;

(6) at least one inert hydrocarbon propellant;

(7) lanolin oil; and

(8) cod liver oil.

The composition has a viscosity and surface tension in a range that allows the composition to be administered to the skin of a user as a pressurized aerosol.

Typically, the substantially anhydrous hydroxylic solvent is an alcohol. A preferred alcohol preparation is SD Alcohol 40.

Typically, the inert hydrocarbon propellant comprises at least one hydrocarbon selected from the group consisting of propane, butane, isobutane, pentane, isopentane, and neopentane. Preferably, the inert hydrocarbon propellant comprises isobutane and propane.

The composition can further comprise other ingredients, such as a cosolvent component and a preservative component.

A preferred composition of this embodiment of the invention comprises:

(1) allantoin in a therapeutically effective quantity;

(2) SD Alcohol 40 in a quantity sufficient to dissolve the allantoin;

(3) propylene glycol;

(4) lanolin oil;

(5) cod liver oil;

(6) propylparaben;

(7) propane; and

(8) isobutane.

In another alternative of this embodiment, the SD Alcohol 40 or other substantially anhydrous hydroxylic solvent, such as another alcohol, is replaced by the same proportion of propylene glycol.

Another aspect of the present invention is a method of treating a skin condition or disease characterized by ulceration, inflammation, or blistering of the skin comprising applying to the skin an allantoin-containing composition according to the present invention in a therapeutically effective quantity. The skin condition or disease can be selected from the group including epidermolysis bullosa, decubitus ulcers, pressure ulcers, diabetic ulcers, milia, burns, eczema, urticaria, atopic dermatitis, contact dermatitis, arthritis, gout, and lupus erythematosus.

Typically, the skin condition or disease is epidermolysis bullosa.

Methods according to the present invention can further comprise administering an additional therapeutic agent in a therapeutically effective quantity. The additional therapeutic agent can be selected from the group including steroids, nonsteroidal anti-inflammatory agents, leukotriene antagonists, and monoclonal antibodies.

### DETAILED DESCRIPTION OF THE PREFERRED EMBODIMENTS

One aspect of the present invention is allantoin-containing compositions that are in the form of gels and that can be administered to patients in that form. One embodiment of an allantoin-containing composition according to this aspect of the present invention comprises:

(1) allantoin in a therapeutically effective quantity;

(2) a complex comprising mineral oil and at least one hydrogenated alkylene copolymer;

(3) lanolin oil; and

(4) cod liver oil.

The composition forms a gel.

Typically, at least one monomer in the hydrogenated alkylene copolymer is styrene. Preferably, the hydrogenated alkylene copolymer includes both a hydrogenated butylene/ethylene/styrene copolymer and a hydrogenated ethylene/propylene/styrene copolymer. Other hydrogenated alkylene copolymers can be used.

The composition can further comprise other ingredients.

For example, the composition can further comprise a solvent component. Typically, the solvent component includes at least one solvent selected from the group consisting of ethylene glycol, propylene glycol, butylene glycol, and glycerin. Preferably, the solvent component is propylene glycol.

The composition can further comprise an antioxidant component to prevent rancidity of ingredients such as cod liver oil. Typically, the antioxidant component comprises at least one antioxidant selected from the group consisting of butylated hydroxytoluene and butylated hydroxyanisole. Preferably, the antioxidant component is butylated hydroxytoluene.

The composition can further comprise a preservative component to increase stability of the composition. Typically, the preservative component comprises at least one preservative selected from the group consisting of methylparaben, ethylparaben, propylparaben, and butylparaben. Preferably, the preservative component is propylparaben.

The composition can further include fragrance. The use of fragrance is well known in the cosmetic art and in the art of over-the-counter drug formulation, and many suitable fragrances are known in the art. The stability and function of the composition are not altered by the presence or absence of fragrance.

Preferably, this embodiment of the composition comprises:

(1) allantoin in a therapeutically effective quantity;

(2) a complex comprising:

(a) mineral oil;

(b) a hydrogenated butylene/ethylene/styrene copolymer; and

(c) a hydrogenated ethylene/propylene/styrene copolymer;

(3) lanolin;

(4) cod liver oil;

(5) butylated hydroxytoluene;

(6) propylparaben;

(7) fragrance; and

(8) propylene glycol.

The following discussion describes ranges, preferred concentrations, and optimum concentrations for preferred compositions according to this embodiment of the present invention. For this and other ranges, preferred concentrations, and optimum concentrations of specific ingredients for other embodiments as given below, all percentages are weight percentages unless otherwise specified.

Allantoin can comprise from 0.5% to 10.0% of this embodiment of the composition. Preferably, allantoin comprises from about 1.125% to about 1.875% of this embodiment of the composition. More preferably, allantoin comprises from about 1.35% to about 1.65% of this embodiment of the composition. An optimum concentration of allantoin is about 1.50% of this embodiment of the composition.

The complex comprising mineral oil, a hydrogenated butylene/ethylene/styrene copolymer, and a hydrogenated ethylene/propylene/styrene copolymer can comprise from about 70.0% to about 90.0% of this embodiment of the composition. Preferably, the complex comprising mineral oil, a hydrogenated butylene/ethylene/styrene copolymer, and a hydrogenated ethylene/propylene/styrene copolymer comprises from about 75.0% to about 90.0% of this embodiment of the composition. More preferably, the complex comprising mineral oil, a hydrogenated butylenelethylene/styrene copolymer and a hydrogenated ethylene/propylene/styrene copolymer comprises from about 82.0% to about 88.0% of this embodiment of the composition. An optimum concentration of the complex comprising mineral oil, a hydrogenated butylene/ethylene/styrene copolymer, and a hydrogenated ethylene/propylene/styrene copolymer is about 84.65% of this embodiment of the composition.

Lanolin oil can comprise from about 3.5% to about 14.0% of this embodiment of the composition. Preferably, lanolin oil comprises from about 5.25% to about 8.75% of this embodiment of the composition. More preferably, lanolin oil comprises from about 6.30% to about 7.70% of this embodiment of the composition. An optimum concentration of lanolin oil is about 7.00% of this embodiment of the composition.

Cod liver oil can comprise from about 1.0% to about 4.0% of this embodiment of the composition. Preferably, cod liver oil comprises from about 1.50% to about 2.50% of this embodiment of the composition. More preferably, cod liver oil comprises from about 1.80% to about 2.20% of this embodiment of the composition. An optimum concentration of cod liver oil is about 2.00% of this embodiment of the composition.

Butylated hydroxytoluene can comprise from about 0.25% to about 1.0% of this embodiment of the composition. Preferably, butylated hydroxytoluene comprises from about 0.375% to about 0.625% of this embodiment of the composition. More preferably, butylated hydroxytoluene comprises from about 0.45% to about 0.55% of this embodiment of the composition. An optimum concentration of butylated hydroxytoluene is about 0.50% of this embodiment of the composition.

Propylparaben can comprise from about 0.075% to about 0.30% of this embodiment of the composition. Preferably, propylparaben comprises from about 0.1125% to about 0.1875% of this embodiment of the composition. More preferably, propylparaben comprises from about 0.135% to about 0.165% of this embodiment of the composition. An optimum concentration of propylparaben is about 0.15% of this embodiment of the composition.

Fragrance can comprise from about 0.10% to about 0.40% of this embodiment of the composition. Preferably, fragrance comprises from about 0.15% to about 0.25% of this embodiment of the composition. More preferably, fragrance comprises from about 0.18% to about 0.22% of this embodiment of the composition. An optimum concentration of fragrance is about 0.20% of this embodiment of the composition.

Propylene glycol can comprise from about 2.0% to about 8.0% of this embodiment of the composition. Preferably, propylene glycol comprises from about 3.0% to about 5.0% of this embodiment of the composition. More preferably, propylene glycol comprises from about 3.60% to about 4.40% of this embodiment of the composition. An optimum concentration of propylene glycol is about 4.00% of this embodiment of the composition.

Another embodiment of this aspect of the present invention is an allantoin-containing composition comprising:

(1) allantoin in a therapeutically effective quantity;

(2) water;

(3) a complex comprising mineral oil and at least one hydrogenated alkylene copolymer;

(4) lanolin oil; and

(5) cod liver oil.

This embodiment of the composition forms a gel.

The complex comprising mineral oil and at least one hydrogenated alkylene copolymer is as described above.

The composition can further comprise other ingredients.

For example, the composition can further comprise a solvent component. Typically, the solvent component comprises at least one solvent selected from the group consisting of ethylene glycol, propylene glycol, butylene glycol, and glycerin. Preferably, the solvent component is propylene glycol.

The composition can further comprise an antioxidant component. Typically, the antioxidant component comprises at least one antioxidant selected from the group consisting of butylated hydroxytoluene and butylated hydroxyanisole. Preferably, the antioxidant is butylated hydroxytoluene.

The composition can further comprise a preservative component. Typically, the preservative component comprises at least one preservative selected from the group consisting of methylparaben, ethylparaben, propylparaben, and butylparaben. Preferably, the preservative component is propylparaben.

The composition can further include fragrance as described above. The stability and function of the composition are not altered by the presence or absence of fragrance.

Preferably, this embodiment of an allantoin-containing composition comprises:

(1) allantoin in an therapeutically effective quantity;

(2) water;

(3) a complex comprising:

(a) mineral oil;

(b) a hydrogenated butylene/ethylene/styrene copolymer; and

(c) a hydrogenated ethylene/propylene/styrene copolymer;

(4) lanolin oil;

(5) cod liver oil;

(6) butylated hydroxytoluene;

(7) propylparaben;

(8) fragrance; and

(9) propylene glycol.

The following discussion describes ranges, preferred concentrations, and optimum concentrations for preferred compositions according to this embodiment of the present invention.

Allantoin can comprise from about 0.5% to about 10.0% of this embodiment of the composition. Preferably, allantoin comprises from about 1.125% to about 1.875% of this embodiment of the composition. More preferably, allantoin comprises from about 1.35% to about 1.65% of this embodiment of the composition. An optimum concentration of allantoin is about 1.50% of this embodiment of the composition.

Water can comprise from about 1.5% to about 6.0% of this embodiment of the composition. Preferably, water comprises from about 2.25% to about 3.75% of this embodiment of the composition. More preferably, water comprises from about 2.70% to about 3.30% of this embodiment of the composition. An optimum concentration of water is about 3.00% of this embodiment of the composition.

The complex comprising mineral oil, a hydrogenated butylene/ethylene/styrene copolymer, and a hydrogenated ethylene/propylene/styrene copolymer can comprise from about 60.0% to about 90.0% of this embodiment of the composition. Preferably, the complex comprising mineral oil, a hydrogenated butylene/ethylene/styrene copolymer, and a hydrogenated ethylene/propylene/styrene copolymer comprises from about 65.0% to about 88.0% of the composition. More preferably, the complex comprising mineral oil, a hydrogenated butylene/ethylene/styrene copolymer, and a hydrogenated ethylene/propylene/styrene copolymer comprises from about 74.0% to about 86.0% of the composition. An optimum concentration of the complex comprising mineral oil, a hydrogenated butylene/ethylene/styrene copolymer, and a hydrogenated ethylene/propylene/styrene copolymer is about 79.60% of this embodiment of the composition.

Lanolin oil can comprise from about 3.5% to about 14.0% of this embodiment of the composition. Preferably, lanolin oil comprises from about 5.25% to about 8.75% of this embodiment of the composition. More preferably, lanolin oil comprises from about 6.30% to about 7.70% of this embodiment of the composition. An optimum concentration of lanolin oil is about 7.00% of this embodiment of the composition.

Cod liver oil can comprise from about 1.0% to about 4.0% of this embodiment of the composition. Preferably, cod liver oil comprises from about 1.50% to about 2.50% of this embodiment of the composition. More preferably, cod liver oil comprises from about 1.80% to about 2.20% of the composition. An optimum concentration of cod liver oil is about 2.00% of this embodiment of the composition.

Butylated hydroxytoluene can comprise from about 0.25% to about 1.0% of this embodiment of the composition. Preferably, butylated hydroxytoluene comprises from about 0.375% to about 0.625% of this embodiment of the composition. More preferably, butylated hydroxytoluene comprises from about 0.45% to about 0.55% of this embodiment of the composition. An optimum concentration of butylated hydroxytoluene is about 0.50% of this embodiment of the composition.

Propylparaben can comprise from about 0.10% to about 0.40% of this embodiment of the composition. Preferably, propylparaben comprises from about 0.15% to about 0.25% of this embodiment of the composition. More preferably, propylparaben comprises from about 0.18% to about 0.22% of this embodiment of the composition. An optimum concentration of propylparaben is about 0.20% of this embodiment of the composition.

Fragrance can comprise from about 0.10% to about 0.40% of this embodiment of the composition. Preferably, fragrance comprises from about 0.15% to about 0.25% of this embodiment of the composition. More preferably, fragrance comprises from about 0.18% to about 0.22% of this embodiment of the composition. An optimum concentration of fragrance is about 0.20% of this embodiment of the composition.

Propylene glycol can comprise from about 3.0% to about 12.0% of this embodiment of the composition. Preferably, propylene glycol comprises from about 4.5% to about 7.5% of this embodiment of the composition. More preferably, propylene glycol comprises from about 5.40% to about 6.60% of this embodiment of the composition. An optimum concentration of propylene glycol is about 6.00% of this embodiment of the composition.

Another embodiment of an allantoin-containing composition according to this aspect of the present invention comprises:

(1) allantoin in a therapeutically effective quantity;

(2) a polymer selected from the group consisting of polyglyceryl methacrylate, polyglyceryl acrylate, and polyglyceryl ethacrylate;

(3) lanolin oil; and

(4) cod liver oil.

The composition forms a gel.

Preferably, the polymer is polyglyceryl methacrylate. However, other similar polymers can alternatively be used.

The composition can further comprise other ingredients.

For example, the composition can further comprise a solvent component. Typically, the solvent component comprises at least one solvent selected from the group consisting of ethylene glycol, propylene glycol, butylene glycol, and glycerin. Preferably, the solvent component is propylene glycol.

The composition can further comprise an antioxidant component. Typically, the antioxidant component comprises at least one antioxidant selected from the group consisting of butylated hydroxytoluene and butylated hydroxyanisole. Preferably, the antioxidant is butylated hydroxytoluene.

The composition can further comprise a preservative component. Typically, the preservative component comprises at least one preservative selected from the group consisting of methylparaben, ethylparaben, propylparaben, and butylparaben. Preferably, the preservative component is propylparaben.

The composition can further include fragrance as described above. The stability and function of the composition are not altered by the presence or absence of fragrance.

Preferably, this embodiment of an allantoin-containing composition comprises:

(1) allantoin in a therapeutically effective quantity;

(2) polyglyceryl methacrylate;

(3) lanolin oil;

(4) cod liver oil;

(5) butylated hydroxytoluene;

(6) propylparaben;

(7) propylene glycol; and

(8) fragrance.

The following discussion describes ranges, preferred concentrations, and optimum concentrations for preferred compositions according to this embodiment of the present invention.

Allantoin can comprise from 0.5% to 10.0% of this embodiment of the composition. Preferably, allantoin comprises from 1.125% to about 1.875% of this embodiment of the composition. More preferably, allantoin comprises from about 1.35% to about 1.65% of this embodiment of the composition. An optimum concentration of allantoin is about 1.50% of this embodiment of the composition.

Polyglyceryl methacrylate can comprise from about 60.0% to about 90.0% of this embodiment of the composition. Preferably, the polyglyceryl methacrylate comprises from about 65.0% to about 85.0% of this embodiment of the composition. More preferably, the polyglyceryl methacrylate comprises from about 66.0% to about 74.0% of this embodiment of the composition. An optimum concentration of polyglyceryl methacrylate is about 70.65% of this embodiment of the composition.

Lanolin oil can comprise from about 3.5% to about 14.0% of this embodiment of the composition. Preferably, lanolin oil comprises from about 5.25% to about 8.75% of the composition. More preferably, lanolin oil comprises from about 6.30% to about 7.70% of this embodiment of the composition. An optimum concentration of lanolin oil is about 7.00% of this embodiment of the composition.

Cod liver oil can comprise from about 1.0% to about 4.0% of this embodiment of the composition. Preferably, cod liver oil comprises from about 1.50% to about 2.50% of this embodiment of the composition. More preferably, cod liver oil comprises from about 1.80% to about 2.20% of this embodiment of the composition. An optimum concentration of cod liver oil is about 2.00% of this embodiment of the composition.

Butylated hydroxytoluene can comprise from about 0.25% to about 1.0% of this embodiment of the composition. Preferably, butylated hydroxytoluene comprises from about 0.375% to about 0.625% of this embodiment of the composition. More preferably, butylated hydroxytoluene comprises from about 0.45% to about 0.55% of this embodiment of the composition. An optimum concentration of butylated hydroxytoluene is about 0.50% of this embodiment of the composition.

Propylparaben can comprise from about 0.075% to about 0.30% of this embodiment of the composition. Preferably, propylparaben comprises from about 0.1125% to about 0.1875% of this embodiment of the composition. More preferably, propylparaben comprises from about 0.135% to about 0.165% of this embodiment of the composition. An optimum concentration of propylparaben is about 0.15% of this embodiment of the composition.

Propylene glycol can comprise from about 9.0% to about 27.0% of this embodiment of the composition. Preferably, propylene glycol comprises from about 13.5% to about 22.5% of this embodiment of the composition. More preferably, propylene glycol comprises from about 16.2% to about 19.8% of this embodiment of the composition. An optimum concentration of propylene glycol is about 18.00% of this embodiment of the composition.

Fragrance can comprise from about 0.10% to about 0.40% of this embodiment of the composition. Preferably, fragrance can comprise from about 0.15% to about 0.25% of this embodiment of the composition. More preferably, fragrance comprises from about 0.18% to about 0.22% of this embodiment of the composition. An optimum concentration of fragrance is about 0.20% of this embodiment of the composition.

Another embodiment of a composition according to this aspect of the present invention comprises:

(1) allantoin in a therapeutically effective quantity;

(2) water;

(3) a polymer selected from the group consisting of polyglyceryl methacrylate, polyglyceryl acrylate, and polyglyceryl ethacrylate;

(4) lanolin oil; and

(5) cod liver oil.

Preferably, the polymer is polyglyceryl methacrylate.

The composition can further comprise other components.

For example, the composition can further comprise a solvent component. Typically, the solvent component comprises at least one solvent selected from the group consisting of ethylene glycol, propylene glycol, butylene glycol, and glycerin. Preferably, the solvent component is propylene glycol.

The composition can further comprise an antioxidant component. Typically, the antioxidant component comprises at least one antioxidant selected from the group consisting of butylated hydroxytoluene and butylated hydroxyanisole. Preferably, the antioxidant is butylated hydroxytoluene.

The composition can further comprise a preservative component. Typically, the preservative component comprises at least one preservative selected from the group consisting of methylparaben, ethylparaben, propylparaben, and butylparaben. Preferably, the preservative component is propylparaben.

The composition can further include fragrance as described above. The stability and function of the composition are not altered by the presence or absence of fragrance.

Preferably, this embodiment of the composition comprises:

(1) allantoin in a therapeutically effective quantity;

(2) water;

(3) polyglyceryl methacrylate;

(4) lanolin oil;

(5) cod liver oil;

(6) butylated hydroxytoluene;

(7) propylparaben;

(8) fragrance; and

(9) propylene glycol.

The following discussion describes ranges, preferred concentrations, and optimum concentrations for preferred compositions according to this embodiment of the present invention.

Allantoin can comprise from about 0.5% to about 10.0% of this embodiment of the composition. Preferably, allantoin comprises from about 1.125% to about 1.875% of this embodiment of the composition. More preferably, allantoin comprises from about 1.35% to about 1.65% of this embodiment of the composition. An optimum concentration of allantoin is about 1.50% of this embodiment of the composition.

Water can comprise from about 1.5% to about 6.0% of this embodiment of the composition. Preferably, water comprises from about 2.25% to about 3.75% of this embodiment of the composition. More preferably, water comprises from about 2.70% to about 3.30% of this embodiment of the composition. An optimum concentration of water is about 3.00% of this embodiment of the composition.

Polyglyceryl methacrylate can comprise from about 55.0% to about 85.0% of this embodiment of the composition. Preferably, polyglyceryl methacrylate comprises from about 60.0% to about 80.0% of this embodiment of the composition. More preferably, polyglyceryl methacrylate comprises from about 66.0% to about 74.0% of this embodiment of the composition. An optimum concentration of polyglyceryl methacrylate is about 67.65% of this embodiment of the composition.

Lanolin oil can comprise from about 3.5% to about 14.0% of this embodiment of the composition. Preferably, lanolin oil comprises from about 5.25% to about 8.75% of this embodiment of composition. More preferably, lanolin oil comprises from about 6.30% to about 7.70% of this embodiment of the composition. An optimum concentration of lanolin oil is about 7.00% of this embodiment of the composition.

Cod liver oil can comprise from about 1.0% to about 4.0% of this embodiment of the composition. Preferably, cod liver oil comprises from about 1.50% to about 2.50% of this embodiment of the composition. More preferably, cod liver oil comprises from about 1.80% to about 2.20% of this embodiment of the composition. An optimum concentration of cod liver oil is about 2.00% of this embodiment of the composition.

Butylated hydroxytoluene can comprise from about 0.25% to about 1.0% of this embodiment of the composition. Preferably, butylated hydroxytoluene comprises from about 0.375% to about 0.625% of this embodiment of the composition. More preferably, butylated hydroxytoluene comprises from about 0.45% to about 0.55% of this embodiment of the composition. An optimum concentration of butylated hydroxytoluene is about 0.50% of this embodiment of the composition.

Propylparaben can comprise from about 0.075% to about 0.30% of this embodiment of the composition. Preferably, propylparaben comprises from about 0.1125% to about 0.1875% of this embodiment of the composition. More preferably, propylparaben comprises from about 0.135% to about 0.165% of this embodiment of the composition. An optimum concentration of propylparaben is about 0.15% of this embodiment of the composition.

Fragrance can comprise from about 0.10% to about 0.40% of this embodiment of the composition. Preferably, fragrance comprises from about 0.15% to about 0.25% of this embodiment of the composition. More preferably, fragrance comprises from about 0.18% to about 0.22% of this embodiment of the composition. An optimum concentration of fragrance is about 0.20% of this embodiment of the composition.

Propylene glycol can comprise from about 9.0% to about 27.0% of this embodiment of the composition. Preferably, propylene glycol comprises from about 13.5% to about 22.5% of this embodiment of the composition. More preferably, propylene glycol comprises from about 16.2% to about 19.8% of this embodiment of the composition. An optimum concentration of propylene glycol is about 18.00% of this embodiment of the composition.

Another embodiment of an allantoin-containing composition according to the present invention comprises:

(1) water;

(2) allantoin in a therapeutically effective quantity;

(3) a carboxypolymethylene polymer;

(4) PEG-100 stearate;

(5) lanolin oil;

(6) cod liver oil; and

(7) a water-soluble base to adjust the pH from about 3.5-6.0.

Typically, the water-soluble base is sodium hydroxide. Preferably the water-soluble base adjusts the pH to about 4.8. Preferably, the carboxypolymethylene polymer is a cross-linked polyacrylic acid that is cross-linked by allylsucrose or allylpentaerythritol with the polyacrylic acid. Such polymers are marketed under the brand names “Carbomer” and “Carbopol.” A suitable carboxypolymethylene polymer is marketed by B.F. Goodrich under the brand name “Carbomer.” This is a slightly cross-linked polyacrylic acid that is from 1% to 2% cross-linked by allylsucrose or allylpentaerythritol with the polyacrylic acid. The resulting molecular weight range of this polymer is from about 2×10⁶ daltons to about 1×10⁹ daltons. The average molecular weight of this polymer is about 4×10⁶ daltons.

The composition can further comprise other ingredients.

For example, the composition can further comprise a solvent component. Typically, the solvent component comprises at least one solvent selected from the group consisting of ethylene glycol, propylene glycol, butylene glycol, and glycerin. Preferably, the solvent is propylene glycol.

The composition can further comprise an antioxidant component. Typically, the antioxidant component comprises at least one antioxidant selected from the group consisting of butylated hydroxytoluene and butylated hydroxyanisole. Preferably, the antioxidant is butylated hydroxytoluene.

The composition can further comprise a preservative component. Typically, the preservative component comprises at least one preservative selected from the group consisting of 2-methyl-4-isothiazolin-3-one and a complex comprising 2-methyl-4isothiazolin-3-one and methylchloroisothiazolinone. Preferably, the preservative is 2methyl-4-isothiazolin-3-one.

Preferably, this embodiment of the composition comprises:

(1) water;

(2) allantoin in a therapeutically effective quantity;

(3) a cross-linked polyacrylic acid that is cross-linked by allylsucrose or allylpentaerythritol with the polyacrylic acid;

(4) PEG-100 stearate;

(5) lanolin oil;

(6) cod liver oil;

(7) sodium hydroxide;

(8) propylene glycol;

(9) butylated hydroxytoluene; and

(10) 2-methyl-4-isothiazolin-3-one.

The following discussion describes ranges, preferred concentrations, and optimum concentrations for preferred compositions according to this embodiment of the present invention.

Water can comprise from about 60.0% to about 90.0% of this embodiment of the present invention. Preferably, water comprises from about 65.0% to about 88.0% of this embodiment of the invention. More preferably, water comprises from about 75.0% to about 85.0% of this embodiment of the composition. An optimum concentration of water is about 78.47% of this embodiment of the composition.

Allantoin can comprise from about 0.5% to about 10.0% of the composition. Preferably, allantoin comprises from about 1.125% to about 1.875% of this embodiment of the composition. More preferably, allantoin comprises from about 1.35% to about 1.65% of this embodiment of the composition. An optimum concentration of allantoin is about 1.50% of this embodiment of the composition.

The cross-linked polyacrylic acid that is cross-linked by allylsucrose or allylpentaerythritol with the polyacrylic acid can comprise from about 0.5% to about 2.0% of this embodiment of the composition. Preferably, the cross-linked polyacrylic acid that is cross-linked by allylsucrose or allylpentaerythritol with the polyacrylic acid is about 0.75% to about 1.25% of this embodiment of the composition. More preferably, the cross-linked polyacrylic acid that is cross-linked by allylsucrose or allylpentaerythritol with the polyacrylic acid comprises from about 0.90% to about 1.10% of this embodiment of the composition. An optimum concentration of the cross-linked polyacrylic acid that is cross-linked by allylsucrose or allylpentaerythritol with the polyacrylic acid is about 1.00% of this embodiment of the composition.

PEG-100 stearate can comprise from about 0.75% to about 3.0% of this embodiment of the composition. Preferably, PEG-100 stearate comprises from about 1.125% to about 1.875% of this embodiment of the composition. More preferably, PEG100 stearate comprises from about 1.35% to about 1.65% of this embodiment of the composition. An optimum concentration of PEG-100 stearate is about 1.50% of this embodiment of the composition.

Lanolin oil can comprise from about 3.5% to about 14.0% of this embodiment of the composition. Preferably, lanolin oil comprises from about 5.25% to about 8.75% of this embodiment of the composition. More preferably, lanolin oil comprises from about 6.30% to about 7.70% of this embodiment of the composition. An optimum concentration of lanolin oil is about 7.00% of this embodiment of the composition.

Cod liver oil can comprise from about 1.0% to about 4.0% of this embodiment of the composition. Preferably, cod liver oil comprises from about 1.50% to about 2.50% of this embodiment of the composition. More preferably, cod liver oil comprises from about 1.80% to about 2.20% of this embodiment of the composition. An optimum concentration of cod liver oil is about 2.00% of this embodiment of the composition.

Sodium hydroxide is present in a quantity sufficient to adjust the pH of the composition.

Propylene glycol can comprise from about 4.0% to about 16.0% of this embodiment of the composition. Preferably, propylene glycol comprises from about 6.0% to about 10.0% of this embodiment of the composition. More preferably, propylene glycol comprises from about 7.20% to about 8.80% of this embodiment of the composition. An optimum concentration of propylene glycol is about 8.00% of this embodiment of the composition.

Butylated hydroxytoluene can comprise from about 0.25% to about 1.0% of this embodiment of the composition. Preferably, butylated hydroxytoluene comprises from about 0.375% to about 0.625% of this embodiment of the composition. More preferably, butylated hydroxytoluene comprises from about 0.45% to about 0.55% of this embodiment of the composition. An optimum concentration of butylated hydroxytoluene is about 0.50% of this embodiment of the composition.

The preservative 2-methyl-4-isothiazolin-3-one can comprise from about 0.015% to about 0.060% of this embodiment of the composition. Preferably, 2-methyl-4-isothiazolin-3one comprises from about 0.225% to about 0.375% of this embodiment of the composition. More preferably, 2-methyl-4-isothiazolin-3-one comprises from about 0.027% to about 0.033% of this embodiment of the composition. An optimum concentration of 2-methyl-4isothiazolin-3-one comprises about 0.030% of this embodiment of the composition. A suitable preparation of 2-methyl-4-isothiazolin-3-one is marketed by Rohm and Haas as Neolone 5000.

Another embodiment of this aspect of the invention is an allantoin-containing composition comprising:

(1) water;

(2) allantoin in a therapeutically effective quantity;

(3) a carboxypolymethylene polymer;

(4) lanolin oil;

(5) cod liver oil;

(6) a complex comprising:

(a) a polymer selected from the group consisting of glycerylpolyacrylate, glycerylpolymethacrylate, and glycerylpolyethacrylate; and

(b) glycerin; and

(7) a weak organic base to adjust the pH.

Preferably, the weak organic base is triethanolamine. Alternatively, other weak organic amine-containing bases can be used, such as, but not limited to, ethanolamine or diethanolamine.

Preferably, the carboxypolymethylene polymer is a cross-linked polyacrylic acid that is cross-linked by allylsucrose or allylpentaerythritol with the polyacrylic acid as described above.

Preferably, the complex comprises glycerylpolyacrylate and glycerin.

This embodiment of the composition can further comprise other components.

For example, the composition can further comprise a solvent component. Typically, the solvent component comprises at least one solvent selected from the group consisting of ethylene glycol, propylene glycol, butylene glycol, and glycerin. Preferably, the solvent is propylene glycol.

The composition can further comprise an antioxidant component. Typically, the antioxidant component comprises at least one antioxidant selected from the group consisting of butylated hydroxytoluene and butylated hydroxyanisole. Preferably, the antioxidant is butylated hydroxytoluene.

The composition can further comprise a preservative component. Typically, the preservative component comprises at least one preservative selected from the group consisting of methylparaben, ethylparaben, propylparaben, and butylparaben. Preferably, the preservative component comprises methylparaben and propylparaben.

The composition can further comprise sodium lauroamphoacetate.

The composition can further include fragrance as described above. The stability and function of the composition are not altered by the presence or absence of fragrance.

Preferably, this embodiment of an allantoin-containing composition comprises:

(1) water;

(2) allantoin in a therapeutically effective quantity;

(3) a cross-linked polyacrylic acid that is cross-linked by allylsucrose or allylpenterytritol with a polyacrylic acid;

(4) lanolin oil;

(5) cod liver oil;

(6) a complex comprising glycerylpolyacrylate and glycerin;

(7) triethanolamine;

(8) propylene glycol;

(9) sodium lauroamphoacetate;

(10) butylated hydroxytoluene;

(11) fragrance;

(12) propylparaben; and

(13) methylparaben.

The following discussion describes ranges, preferred concentrations, and optimum concentrations for preferred compositions according to this embodiment of the present invention.

Water can comprise from about 50.0% to about 80.0% of this embodiment of the invention. Preferably, water comprises from about 52.0% to about 70.0% of this embodiment of the invention. More preferably, water comprises from about 55.0% to about 65.0% of this embodiment of the invention. An optimum concentration of water is about 59.15% of this embodiment of the invention.

Allantoin can comprise from about 0.5% to about 10.0% of this embodiment of the invention. Preferably, allantoin comprises from about 1.125% to about 1.875% of this embodiment of the invention. More preferably, allantoin comprises from about 1.35% to about 1.65% of this embodiment of the invention. An optimum concentration of allantoin is about 1.50% of this embodiment of the invention.

The cross-linked polyacrylic acid that is cross-linked by allylsucrose or allylpentaerythritol with the polyacrylic acid can comprise from about 0.15% to about 0.60% of the composition. Preferably, the cross-linked polyacrylic acid that is cross-linked by allylsucrose or allyipentaerythritol with the polyacrylic acid comprises from about 0.225% to about 0.225% to about 0.375% of this embodiment of the composition. More preferably, the cross-linked polyacrylic acid that is cross-linked by allylsucrose or allylpentaerythritol with the polyacrylic acid comprises about 0.27% to about 0.33% of this embodiment of the composition. An optimum concentration of the cross-linked polyacrylic acid that is cross-linked by allylsucrose or allylpentaerythritol with the polyacrylic acid is about 0.30% of this embodiment of the composition.

Lanolin oil can comprise from about 3.5% to about 14.0% of this embodiment of the composition. Preferably, lanolin oil comprises from about 5.25% to about 8.75% of this embodiment of the composition. More preferably, lanolin oil comprises from about 6.30% to about 7.70% of this embodiment of the composition. An optimum concentration of lanolin oil is about 7.00% of this embodiment of the composition.

Cod liver oil can comprise from about 1.0% to about 4.0% of this embodiment of the composition. Preferably, cod liver oil comprises from about 1.50% to about 2.50% of this embodiment of the composition. More preferably, cod liver oil comprises from about 1.80% to about 2.20% of this embodiment of the composition. An optimum concentration of cod liver oil is about 2.00% of this embodiment of the composition.

The complex comprising glycerylpolyacrylate and glycerin can comprise from about 10.0% to about 30.0% of this embodiment of the composition. Preferably, the complex comprising glycerylpolyacrylate and glycerin comprises from about 15.0% to about 25.0% of this embodiment of the composition. More preferably, the complex comprising glycerylpolyacrylate and glycerin comprises from about 18.0% to about 22.0% of this embodiment of the composition. An optimum concentration of the complex comprising glycerylpolyacrylate and glycerin is about 20.00% of this embodiment of the composition.

Triethanolamine can comprise from about 0.135% to about 0.54% of this embodiment of the composition. Preferably, triethanolamine comprises from about 0.2025% to about 0.3375% of this embodiment of the composition. More preferably, triethanolamine comprises from about 0.243% to about 0.297% of this embodiment of the composition. An optimum concentration of triethanolamine is about 0.27% of this embodiment of the composition.

Propylene glycol can comprise from about 4.0% to about 16.0% of this embodiment of the composition. Preferably, propylene glycol comprises from about 6.0% to about 10.0% of this embodiment of the composition. More preferably, propylene glycol comprises from about 7.20% to about 8.80% of this embodiment of the composition. An optimum concentration of propylene glycol is about 8.00% of this embodiment of the composition.

Sodium lauroamphoacetate acetate could comprise from about 0.5% to about 2.0% of this embodiment of the composition. Preferably, sodium lauroamphoacetate comprises from about 0.75% to about 1.25% of this embodiment of the composition. More preferably, sodium lauroamphoacetate comprises from about 0.90% to about 1.10% of this embodiment of the composition. An optimum concentration of sodium lauroamphoacetate comprises about 1.00% of this embodiment of the composition.

Butylated hydroxytoluene can comprise from about 0.25% to about 1.0% of this embodiment of the composition. Preferably, butylated hydroxytoluene comprises from about 0.375% to about 0.625% of this embodiment of the composition. More preferably, butylated hydroxytoluene comprises from about 0.45% to about 0.55% of this embodiment of the composition. An optimum concentration of butylated hydroxytoluene is about 0.50% of this embodiment of the composition.

Fragrance can comprise from about 0.10% to about 0.40% of this embodiment of the composition. Preferably, fragrance comprises from about 0.15% to about 0.25% of this embodiment of the composition. More preferably, fragrance comprises from about 0.18% to about 0.22% of this embodiment of the composition. An optimum concentration of fragrance is about 0.20% of this embodiment of the composition.

Propylparaben can comprise from about 0.075% to about 0.30% of this embodiment of the composition. Preferably, propylparaben comprises from about 0.1125% to about 0.1875% of this embodiment of the composition. More preferably, propylparaben comprises from about 0.135% to about 0.165% of this embodiment of the composition. An optimum concentration of propylparaben is about 0.15% of this embodiment of the composition.

Methylparaben can comprise from about 0.10% to about 0.40% of this embodiment of the composition. Preferably, methylparaben comprises from about 0.15% to about 0.25% of this embodiment of the composition. More preferably, methylparaben comprises from about 0.18% to about 0.22% of this embodiment of the composition. An optimum concentration of methylparaben is about 0.20% of this embodiment of the composition.

Another aspect of the present invention is allantoin-containing compositions that are suitable for administration as aerosols.

One embodiment of an allantoin-containing composition according to the aspect of the present invention comprises:

(1) water;

(2) allantoin in a therapeutically effective quantity;

(3) an anionic ethoxylated surfactant;

(4) lanolin oil; and

(5) cod liver oil; and

(6) an acid to adjust the pH to a value in the range of from about 3.5 to about 6.0.

The composition has a viscosity and surface tension in a range that allows the composition to be administered to the skin of a user as an aerosol by a mechanical spray pump. The use of a mechanical spray pump to deliver cosmetics, medications, and other preparations to the skin is well known in the art and need not be described further here. Typically, in use, such pumps are compressed by the user, as by pushing down a plunger, to create pressure and force the composition to be delivered through a small-diameter nozzle to create an aerosol.

Typically, the anionic ethoxylated surfactant is selected from the group consisting of sodium trideceth sulfate, sodium dodeceth sulfate, ammonium trideceth sulfate, lauryl sulfosuccinate, ethoxylated sodium dodecylbenzene sulfonate, and ethoxylated sodium N-lauryl sarcosinate. Preferably, the anionic ethoxylated surfactant is sodium trideceth sulfate. Other anionic ethoxylated surfactants can be used.

The acid can be a water-soluble organic acid or an inorganic acid. Typically, the acid is a weak organic acid selected from the group consisting of citric acid, ascorbic acid, glycolic acid, lactic acid, benzoic acid, and salicylic acid. Preferably, the acid is citric acid. A suitable inorganic acid is phosphoric acid.

The composition can further comprise other ingredients.

For example, the composition can further comprise a solvent component. Typically, the solvent component includes at least one solvent selected from the group consisting of ethylene glycol, propylene glycol, butylene glycol, and glycerin. Preferably, the solvent component is propylene glycol.

The composition can further comprise an antioxidant component to prevent rancidity of ingredients such as cod liver oil. Typically, the antioxidant component comprises at least one antioxidant selected from the group consisting of butylated hydroxytoluene and butylated hydroxyanisole. Preferably, the antioxidant component is butylated hydroxytoluene.

The composition can further comprise a preservative component. Typically, the preservative component comprises at least one preservative selected from the group consisting of 2-methyl-4-isothiazolin-3-one and a complex comprising 2-methyl-4-isothiazolin-3-one and methylchloroisothiazolinone.

Preferably, the preservative component comprises 2-methyl-4-isothiazolin-3-one, marketed by Rohm and Haas as Neolone 5000.

The composition can further include fragrance. The use of fragrance is well known in the cosmetic art and in the art of over-the-counter drug formulation, and many suitable fragrances are known in the art. The stability and function of the composition are not altered by the presence or absence of fragrance.

Preferably, this embodiment of the composition comprises:

(1) water;

(2) allantoin in a therapeutically effective quantity;

(3) sodium trideceth sulfate;

(4) lanolin oil;

(5) cod liver oil;

(6) butylated hydroxytoluene;

(7) fragrance;

(8) 2-methyl-4-isothiazolin-3-one;

(9) propylene glycol; and

(10) citric acid.

The following discussion describes ranges, preferred concentrations, and optimum concentrations for preferred compositions according to this embodiment of the present invention. For this and other ranges, preferred concentrations, and optimum concentrations of specific ingredients for other embodiments as given below, all percentages are weight percentages unless otherwise specified.

Water can comprise from about 50.0% to about 90.0% of this embodiment of the composition. Preferably, water comprises from about 60.0% to about 80.0% of this embodiment of the composition. More preferably, water comprises from about 65.0% to about 75.0% of this embodiment of the composition. An optimum concentration of water is about 72.77% of this embodiment of the composition.

Allantoin can comprise from about 0.50% to about 10.0% of this embodiment of the composition. Preferably, allantoin can comprise from about 1.0% to about 2.0% of this embodiment of the composition. More preferably, allantoin can comprise from about 1.35% to about 1.65% of this embodiment of the composition. An optimum concentration of allantoin is about 1.50% of this embodiment of the composition.

Sodium trideceth sulfate can comprise from about 0.50% to about 3.0% of the composition. Preferably, sodium trideceth sulfate comprises from about 0.75% to about 1.25% of this embodiment of the composition. More preferably, sodium trideceth sulfate comprises from about 0.90% to about 1.10% of the composition. An optimum concentration of sodium trideceth sulfate is about 1.00% of this embodiment of the composition.

Lanolin oil can comprise from about 2.0% to about 15.0% of the composition. Preferably, lanolin oil comprises from about 5.25% to about 8.75% of this embodiment of the composition. More preferably, lanolin oil comprises from about 6.30% to about 7.70% of the composition. An optimum concentration of lanolin oil is about 7.00% of this embodiment of the composition.

Cod liver oil can comprise from about 1.0% to about 3.0% of this embodiment of the composition. Preferably, cod liver oil comprises from about 1.50% to about 2.50% of this embodiment of the composition. More preferably, cod liver oil comprises from about 1.80% to about 2.20% of this embodiment of the composition. An optimum concentration of cod liver oil is about 2.00% of this embodiment of the composition.

Butylated hydroxytoluene can comprise from about 0.10% to about 1.0% of this embodiment of the composition. Preferably, butylated hydroxytoluene comprises from about 0.375% to about 0.625% of this embodiment of the composition. More preferably, butylated hydroxytoluene comprises from about 0.45% to about 0.55% of this embodiment of the composition. An optimum concentration of butylated hydroxytoluene is about 0.50% of this embodiment of the composition.

Citric acid can comprise from about 0.01% to about 0.10% of this embodiment of the composition. Sufficient citric acid is used to result in a final pH of about 4.0 to about 6.0. Preferably, citric acid comprises from about 0.02% to about 0.08% of this embodiment of the composition. More preferably, citric acid comprises from about 0.025% to about 0.040% of the composition. An optimum concentration of citric acid is about 0.035%

Fragrance can comprise from about 0.05% to about 0.50% of this embodiment of the composition. Preferably, fragrance comprises from about 0.15% to about 0.25% of this embodiment of the composition. More preferably, fragrance comprises from about 0.18% to about 0.22% of this embodiment of the composition. An optimum concentration of fragrance is about 0.20% of this embodiment of the composition.

The preservative 2-methyl-4-isothiazolin-3-one can comprise from about 0.01% to about 0.10% of this embodiment of the composition. Preferably, 2-methyl-4-isothiazolin-3one comprises from about 0.0225% to about 0.0375% of this embodiment of the composition. More preferably, 2-methyl-4-isothiazolin-3-one comprises from about 0.027% to about 0.033% of this embodiment of the composition. An optimum concentration of 2methyl-4-isothiazolin-3-one is about 0.030% of this embodiment of the composition.

Propylene glycol can comprise from about 5.0% to about 30.0% of this embodiment of the composition. Preferably, propylene glycol comprises from about 11.25% to about 18.75% of this embodiment of the composition. More preferably, propylene glycol comprises from about 13.50% to about 16.50% of this embodiment of the composition. An optimum concentration of propylene glycol is about 15.00% of this embodiment of the composition.

Another embodiment of this aspect of the present invention is an allantoin-containing composition comprising:

(1) allantoin in a therapeutically effective quantity;

(2) a substantially anhydrous hydroxylic solvent in a quantity sufficient to dissolve the allantoin;

(3) at least one inert hydrocarbon propellant;

(4) lanolin oil; and

(5) cod liver oil.

The composition has a viscosity and surface tension in a range that allows the composition to be administered to the skin of a user as a pressurized aerosol. The delivery of compositions as pressurized aerosols to the skin is well-known in a variety of applications, and need not be described further in detail here. Typically, such compositions are packaged in a sealed container containing an appropriate propellant; the composition is under pressure in the container. The container typically includes a valve operated by the user, such as by pressing a button. When the valve is opened, the composition passes through a small diameter nozzle to create an aerosol.

Typically, the substantially anhydrous hydroxylic solvent is an alcohol. A preferred alcohol preparation is SD Alcohol 40. Other denatured alcohol preparations can be used.

In another alternative of this embodiment, the SD Alcohol 40 or other substantially anhydrous hydroxylic solvent, such as another alcohol, is replaced by the same proportion of propylene glycol. The propylene glycol is present in a quantity sufficient to dissolve the allantoin. This alternative is shown in Example 3, below. This may be advantageous in reducing irritation and stinging of the skin compared to SD Alcohol 40 alone.

Typically, the inert hydrocarbon propellant comprises at least one hydrocarbon selected from the group consisting of propane, butane, isobutane, pentane, isopentane, and neopentane. Preferably, the inert hydrocarbon propellant comprises isobutane and propane.

The composition can further comprise a cosolvent component. Preferably, the cosolvent component comprises at least one cosolvent selected from the group consisting of ethylene glycol, propylene glycol, butylene glycol, and glycerol. Preferably, the cosolvent component is propylene glycol.

The composition can further comprise a preservative component. The preservative component can comprise at least one preservative selected from the group consisting of methylparaben, ethylparaben, propylparaben, and butylparaben. Preferably, the preservative is propylparaben.

Preferably, this embodiment of an allantoin-containing composition comprises:

(1) allantoin in a therapeutically effective quantity;

(2) SD Alcohol 40 in a quantity sufficient to dissolve the allantoin;

(3) propylene glycol;

(4) lanolin oil;

(5) cod liver oil;

(6) propylparaben;

(7) propane; and

(8) isobutane.

The following discussion describes ranges, preferred concentrations, and optimum concentrations for preferred compositions according to this embodiment of the present invention.

Allantoin can comprise from about 0.50% to about 10.0% of this embodiment of the composition. Preferably, allantoin comprises from about 1.0% to about 2.0% of this embodiment of the composition. More preferably, allantoin comprises from about 1.35% to about 1.65% of this embodiment of the composition. An optimum concentration of allantoin is about 1.50% of this embodiment of the composition.

SD Alcohol 40 comprises from about 15.675% to about 62.70% of this embodiment of the composition. Preferably, SD Alcohol 40 comprises from about 23.5125% to about 39.1875% of this embodiment of the composition. More preferably, SD Alcohol 40 comprises from about 28.215% to about 34.485% of this embodiment of the composition. An optimum concentration of SD Alcohol 40 is about 31.35% of this embodiment of the composition.

Propylene glycol can comprise from about 7.50% to about 30.0% of this embodiment of the composition. Preferably, propylene glycol comprises from about 11.25% to about 18.75% of this embodiment of the composition. More preferably, propylene, glycol comprises from about 13.5% to about 16.5% of this embodiment of the composition. An optimum concentration of propylene glycol is about 15.00% of this embodiment of the composition.

Lanolin oil can comprise from about 2.50% to about 10.0% of this embodiment of the composition. Preferably, lanolin oil comprises from about 3.75% to about 6.25% of this embodiment of the composition. More preferably, lanolin oil comprises from about 4.50% to about 5.50% of this embodiment of the composition. An optimum concentration of lanolin oil is about 5.00% of this embodiment of the composition.

Cod liver oil can comprise from about 1.0% to about 4.0% of this embodiment of the composition. Preferably, cod liver oil comprises from about 1.50% to about 2.50% of this embodiment of the composition. More preferably, cod liver oil comprises from about 1.80% to about 2.20% of this embodiment of the composition. An optimum concentration of cod liver oil is about 2.00% of this embodiment of the composition.

Propylparaben can comprise from about 0.075% to about 0.30% of this embodiment of the composition. Preferably, propylparaben comprises from about 0.1125% to about 0.1875% of this embodiment of the composition. More preferably, propylparaben comprises from about 0.135% to about 0.165% of this embodiment of the composition. An optimum concentration of propylparaben is about 0.15% of this embodiment of the composition.

Propane can comprise from about 11.25% to about 45.0% of this embodiment of the composition. Preferably, propane comprises from about 16.875% to about 28.125% of this embodiment of the composition. More preferably, propane comprises from about 20.25% to about 24.75% of this embodiment of the composition. An optimum concentration of propane is about 22.50% of this embodiment of the composition.

Butane can comprise from about 11.25% to about 45.0% of this embodiment of the composition. Preferably, butane comprises from about 16.875% to about 28.125% of this embodiment of the composition. More preferably, butane comprises from about 20.25% to about 24.75% of this embodiment of the composition. An optimum concentration of butane is about 22.50% of this embodiment of the composition.

If the substantially anhydrous hydroxylic solvent, such as an alcohol like SD Alcohol 40, is omitted and replaced by the same proportion of propylene glycol, preferably, this alternative of the embodiment comprises:

(1) allantoin in a therapeutically effective quantity;

(2) propylene glycol;

(3) lanolin oil;

(4) cod liver oil;

(5) propylparaben;

(6) propane; and

(7) isobutane.

The following discussion describes ranges, preferred concentrations, and optimum concentrations for preferred compositions according to this embodiment of the present invention.

Allantoin can comprise from about 0.50% to about 10.0% of this embodiment of the composition. Preferably, allantoin comprises from about 1.0% to about 2.0% of this embodiment of the composition. More preferably, allantoin comprises from about 1.35% to about 1.65% of this embodiment of the composition. An optimum concentration of allantoin is about 1.50% of this embodiment of the composition.

Propylene glycol can comprise from about 23.175% to about 92.70% of this embodiment of the composition. Preferably, propylene glycol comprises from about 34.7625% to about 57.9375% of this embodiment of the composition. More preferably, propylene glycol comprises from about 41.715% to about 50.985% of this embodiment of the composition. An optimum concentration of propylene glycol is about 46.35% of this embodiment of the composition.

Lanolin oil can comprise from about 2.50% to about 10.0% of this embodiment of the composition. Preferably, lanolin oil comprises from about 3.75% to about 6.25% of this embodiment of the composition. More preferably, lanolin oil comprises from about 4.50% to about 5.50% of this embodiment of the composition. An optimum concentration of lanolin oil is about 5.00% of this embodiment of the composition.

Cod liver oil can comprise from about 1.0% to about 4.0% of this embodiment of the composition. Preferably, cod liver oil comprises from about 1.50% to about 2.50% of this embodiment of the composition. More preferably, cod liver oil comprises from about 1.80% to about 2.20% of this embodiment of the composition. An optimum concentration of cod liver oil is about 2.00% of this embodiment of the composition.

Propylparaben can comprise from about 0.075% to about 0.30% of this embodiment of the composition. Preferably, propylparaben comprises from about 0.1125% to about 0.1875% of this embodiment of the composition. More preferably, propylparaben comprises from about 0.135% to about 0.165% of this embodiment of the composition. An optimum concentration of propylparaben is about 0.15% of this embodiment of the composition.

Propane can comprise from about 11.25% to about 45.0% of this embodiment of the composition. Preferably, propane comprises from about 16.875% to about 28.125% of this embodiment of the composition. More preferably, propane comprises from about 20.25% to about 24.75% of this embodiment of the composition. An optimum concentration of propane is about 22.50% of this embodiment of the composition.

Butane can comprise from about 11.25% to about 45.0% of this embodiment of the composition. Preferably, butane comprises from about 16.875% to about 28.125% of this embodiment of the composition. More preferably, butane comprises from about 20.25% to about 24.75% of this embodiment of the composition. An optimum concentration of butane is about 22.50% of this embodiment of the composition.

Compositions according to the present invention can contain other, optional ingredients. For example, compositions according to the present invention can contain lipid-soluble components such as, but not limited to, caprylic/capric triglycerides; steareth-2; steareth-21; polyglyceryl-3 beeswax; a branched-carboxylic acid ester of a branched-chain alcohol selected from the group consisting of isononyl isononanoate, isodecyl isononanoate, isooctyl isononanoate, isononyl isooctanoate, isodecyl isooctanonoate, isooctyl isooctanoate, isononyl isodecanoate, isooctyl isodecanoate, and isodecyl isodecanoate; an acrylates/C₁₀-C₃₀ alkyl acrylates cross-polymer; methylgluceth-20; a glyceryl ester of a long-chain fatty acid selected from the group consisting of glyceryl monostearate, glyceryl monopalmitate, and glyceryl monoarachidate; hydrogenated vegetable oil; squalane; C₁₂-C₁₅ alkyl benzoates; di-C₁₂-C₁₅ alkyl fumarate; cholesterol; lanolin alcohol; octyldodecanol; isostearic acid; a branched-chain neopentanoate selected from the group consisting of octyldodecyl neopentanoate, heptyldodecyl neopentanoate, nonydodecyl neopentanoate, octylundecyl neopentanoate, heptylundecyl neopentanoate, nonylundecyl neopentanoate, octyltridecyl neopentanoate, heptyltridecyl neopentanoate, and nonyltridecyl neopentanoate; an arachidyl ester of a short-chain carboxylic acid selected from the group consisting of arachidyl propionate, arachidyl acetate, arachidyl butyrate, and arachidyl isobutyrate; a long-chain fatty acid ester of a medium-chain alcohol selected from the group consisting of octyl palmitate, octyl myristate, octyl stearate, heptyl palmitate, heptyl myristate, heptyl stearate, nonyl palmitate, nonyl myristate, and nonyl stearate; jojoba oil; a myristyl ester of a long-chain fatty acid selected from the group consisting of myristyl myristate, myristyl laurate, and myristyl palmitate; bisabolol; hydrogenated jojoba oil; jojoba esters; methylgluceth-20 sesquistearate; PPG-14 butyl ether; PPG-15 stearyl ether; PPG-1-isoceteth-3-acetate; laureth-2-benzoate; diisostearyl dimer dilinoleate; a long-chain cis-monounsaturated fatty acid ester of a medium-chain alcohol; a medium-chain saturated carboxylic acid ester of a long-chain alcohol; hydrogenated soy glycerides; a long-chain fatty acid ester of cetyl alcohol selected from the group consisting of cetyl palmitate, cetyl stearate, and cetyl myristate; palm kernel oil; palm oil; and an arachidyl ester selected from the group consisting of arachidyl acetate, arachidyl propionate, arachidyl butyrate, and arachidyl isobutyrate.

Optionally, the composition can further comprise other ingredients that are generally used in the cosmetic art and in the art of over-the-counter skin preparations. These ingredients include, but are not limited to:

(1) plant extracts, such as, but not limited to: chamomile extract, witch hazel extract, St. John's wort extract, arnica extract, horsetail extract, horse chestnut extract, rose extract, or lavender extract;

(2) a short-chain carboxylic acid ester of tocopherol selected from the group consisting of tocopheryl acetate, tocopheryl propionate, tocopheryl butyrate, and tocopheryl isobutyrate;

(3) a long-chain fatty acid ester of ascorbic acid selected from the group consisting of ascorbyl myristate, ascorbyl palmitate, and ascorbyl stearate;

(4) a long-chain fatty acid ester of retinol or a retinol derivative or analogue wherein the acyl moiety of the ester is selected from the group consisting of myristic acid, palmitic acid, and stearic acid; and

(5) a sunscreen, which can be at least one compound selected from the group consisting of octyl methoxycinnamate, p-aminobenzoic acid, ethyl p-aminobenzoate, isobutyl p-aminobenzoate, glyceryl p-aminobenzoate, p-dimethylaminobenzoic acid, methyl anthranilate, menthyl anthranilate, phenyl anthranilate, benzyl anthranilate, phenylethyl anthranilate, linalyl anthranilate, terpinyl anthranilate, cyclohexenyl anthranilate, amyl salicylate, phenyl salicylate, benzyl salicylate, menthyl salicylate, glyceryl salicylate, dipropyleneglycol salicylate, methyl cinnamate, benzyl cinnamate, α-phenyl cinnamonitrile, butyl cinnamoylpyruvate, umbelliferone, methylacetoumbelliferone, esculetin, methylesculetin, daphnetin, esculin, daphnin, diphenylbutadiene, stilbene, dibenzalacetone, benzalacetophenone, sodium 2-naphthol-3,6-disulfonate, sodium 2-naphthol-6,8-disulfonate, dihydroxynaphthoic acid, salts of dihydroxynaphthoic acid, o-hydroxybiphenyldisulfonates, p-hydroxybiphenyldisulfonates, 7-hydroxycoumarin, 7-methylcoumarin, 3-phenylcoumarin, 2-acetyl-3-bromoindazole, phenylbenzoxazole, methylnaphthoxazole, arylbenzothiazoles, quinine bisulfate, quinine sulfate, quinine chloride, quinine oleate, quinine tannate, 8-hydroxyquinoline salts, 2-phenylquinoline, hydroxy-substituted benzophenones, methoxy-substituted benzophenones, uric acid, vilouric acid, tannic acid, tannic acid hexaethylether, hydroquinone, oxybenzone, sulisobenzone, dioxybenzone, benzoresorcinol, 2,2′,4,4′-tetrahydroxybenzophenone, 2,2′-dihydroxy-4,4′-dimethoxybenzophenone, octabenzone, 4-isopropyldibenzoylmethane, butylmethoxydibenzoylmethane, etocrylene, and 4-isopropyldibenzoylmethane.

Other ingredients can also optionally be included, such as colorants, pigments, and the like. The other ingredients to be used can be chosen depending on the desired appearance of the composition; the ingredients chosen are compatible with the formulation of the composition as a gel or alternatively are compatible with the formulation of the composition as an aerosol and with the required viscosity of the composition for that rate of application.

Compositions according to the present invention can be prepared by standard mixing techniques, such as are conventional in the cosmetic art and in the art of over-the counter drug formulations for blending lipid-soluble components and water-soluble components. These mixing techniques include both manual and mechanical mixing, and include homogenization mixing and propeller and sweep mixing. The mixing techniques to be used can be chosen by one of ordinary skill in the art based on variables such as the viscosity of the components to be mixed and the volume of those components, as well as the relative proportion of lipid-soluble and water-soluble ingredients in those embodiments of the composition that include water or water-soluble ingredients. The compositions can be mixed in two or more batches, such as one batch containing lipid-soluble ingredients and another batch containing water-soluble ingredients, and the batches can then be mixed at the final stage of preparation. These methods can be altered or modified according to the particular ingredients in the composition, such as in those cases where the composition is anhydrous or substantially anhydrous.

Another aspect of the present invention is a method of treating a skin condition or disease characterized by ulceration, inflammation, or blistering of the skin comprising applying to the skin an allantoin-containing composition according to the present invention in a therapeutically effective quantity. The skin condition or disease can be selected from the group including epidermolysis bullosa, decubitus ulcers, pressure ulcers, diabetic ulcers, milia, burns, eczema, urticaria, atopic dermatitis, contact dermatitis, arthritis, gout, and lupus erythematosus.

Typically, the skin condition or disease is epidermolysis bullosa.

Methods according to the present invention can further comprise administering an additional therapeutic agent in a therapeutically effective quantity. The additional therapeutic agent can be selected from the group consisting of steroids, nonsteroidal anti-inflammatory agents, leukotriene antagonists, and monoclonal antibodies. These other therapeutic agents can either be applied topically to the skin or can be administered systemically, such as orally, intravenously, or by other conventional routes that are generally known in the art. Additional agents can be administered to promote healing in the form of conventional creams or emulsions.

The dosages of the allantoin-containing compositions to be administered and the frequency of those dosages can be determined by one of ordinary skill in the art depending on the particular disease affecting the patient, the clinical severity of the disease, the age and weight of the patient, the exposure of the patient to conditions that may precipitate outbreaks of dermatological or systemic inflammatory conditions, the degree of exposure to environmental insults, other drugs being administered, the response of the patient, and other pharmacokinetic factors generally understood in the art, such as liver and kidney metabolism. The interrelationship of dosages for animals of various sizes and species and humans based on mg/m³ surface area as described in E. J. Freireich et al., “Quantitative Comparison of Toxicity of Anti Cancer Agents in Mouse, Rat, Hamster, Dog, Monkey, and Man,” Cancer Chemother. Rep. 50:219-244 (1966). Adjustments in the dosage regimen can be made to optimize the therapeutic response. Doses can be divided and administered on a daily basis or the dose can be reduced proportionately depending upon the therapeutic situation.

The allantoin-containing composition can be administered from once per day up to at least five times per day depending on the severity of the disease, the total dosage to be administered, and the judgment of the treating physician. In some cases, the allantoin-containing composition need not be administered on a daily basis, but can be administered every other day, every third day, or on other such schedules. However, it is generally preferred to administer the allantoin-containing composition daily.

The invention is illustrated by the following examples. These examples are for illustrative purposes only and are not intended to limit the invention.

### EXAMPLES

### Example 1

### Preparation of Allantoin-Containing Composition in Gel Form with Complex Comprising Mineral Oil Hydrogenated Butylene/Ethylene/Styrene Copolymer and Hydrogenated Butylene/Propylene/Styrene Copolymer

A substantially anhydrous allantoin-containing composition in gel form with a complex comprising mineral oil, a hydrogenated butylene/ethylene/styrene copolymer, and a hydrogenated butylene/propylene/styrene copolymer is prepared as shown in Table 1.

TABLE 1
ALLANTOIN-CONTAINING
COMPOSITION IN GEL FORM WITH COMPLEX
COMPRISING MINERAL OIL, HYDROGENATED BUTYLENE/
ETHYLENE/STYRENE COPOLYMER, AND HYDROGENATED
ETHYLENE/PROPYLENE/STYRENE COPOLYMER

| | | | MORE- | |
|---|---|---|---|---|
| | | | HIGHLY | OPTI- |
| INGREDIENT | RANGE | PREFERRED | PREFERRED | MUM |
| Allantoin | 0.5-10.0 | 1.125-1.875 | 1.35-1.65 | 1.50 |
| Complex with | 70.0-90.0 | 75.0-90.0 | 82.0-88.0 | 84.65 |
| Oil and | | | | |
| Copolymer | | | | |
| Lanolin Oil | 3.5-14.0 | 5.25-8.75 | 6.30-7.70 | 7.00 |
| Cod Liver Oil | 1.0-4.0 | 1.50-2.50 | 1.80-2.20 | 2.00 |
| Butylated | 0.25-1.0 | 0.375-0.625 | 0.45-0.55 | 0.50 |
| Hydroxy- | | | | |
| toluene | | | | |
| Propylparaben | 0.075-0.30 | 0.1125-0.1875 | 0.135-0.165 | 0.15 |
| Fragrance | 0.10-0.40 | 0.15-0.25 | 0.18-0.22 | 0.20 |
| Propylene | 2.0-8.0 | 3.0-5.0 | 3.60-4.40 | 4.00 |
| Glycol | | | | |

### Example 2

### Preparation of Complex Comprising Mineral Oil, Hydrogenated Butylene/Ethylene/Styrene Copolymer, and Hydrogenated Butylene/Propylene/Styrene Copolymer

An aqueous allantoin-containing composition in gel form with a complex comprising mineral oil, a hydrogenated butylene/ethylene/styrene copolymer, and a hydrogenated butylene/propylene/styrene copolymer is prepared as shown in Table 2.

TABLE 2
AQUEOUS ALLANTOIN-CONTAINING
COMPOSITION IN GEL FORM WITH COMPLEX
COMPRISING MINERAL OIL, HYDROGENATED BUTYLENE/
ETHYLENE/STYRENE COPOLYMER, AND HYDROGENATED
ETHLYLENE/PROPYLENE/STYRENE COPOLYMER

| | | | MORE | |
|---|---|---|---|---|
| | | | HIGHLY | OPTI- |
| INGREDIENT | RANGE | PREFERRED | PREFERRED | MUM |
| Allantoin | 0.5-10.0 | 1.125-1.875 | 1.35-1.65 | 1.50 |
| Water | 1.5-6.0 | 2.25-3.75 | 2.70-3.30 | 3.00 |
| Complex with | 60.0-90.0 | 65.0-88.0 | 74.0-86.0 | 79.60 |
| Mineral Oil | | | | |
| and Copolymer | | | | |
| Lanolin Oil | 3.5-14.0 | 5.25-8.75 | 6.30-7.70 | 7.00 |
| Cod Liver Oil | 1.0-4.0 | 1.50-2.50 | 1.80-2.20 | 2.00 |
| Butylated | 0.25-1.0 | 0.375-0.625 | 0.45-0.55 | 0.50 |
| Hydroxytoluene | | | | |
| Propylparaben | 0.10-0.40 | 0.15-0.25 | 0.18-0.22 | 0.20 |
| Fragrance | 0.10-0.40 | 0.15-0.25 | 0.18-0.22 | 0.20 |
| Propylene Glycol | 3.0-12.0 | 4.50-7.50 | 5.40-6.60 | 6.00 |

### Example 3

### Preparation of Allantoin-Containing Composition in Gel Form with Polyalyceryl Methacrylate Polymer

A substantially anhydrous allantoin-containing composition in gel form with a polyglyceryl methacrylate polymer is prepared as shown in Table 3.

TABLE 3
ALLANTOIN-CONTAINING COMPOSITION IN GEL
FORM WITH POLYGLYCERYL METHACRYLATE POLYMER

| | | | MORE | |
|---|---|---|---|---|
| | | | HIGHLY | OPTI- |
| INGREDIENT | RANGE | PREFERRED | PREFERRED | MUM |
| Allantoin | 0.5-10.0 | 1.125-1.875 | 1.35-1.65 | 1.50 |
| Polyglyceryl | 60.0-90.0 | 65.0-85.0 | 66.0-74.0 | 70.65 |
| Methacrylate | | | | |
| Lanolin Oil | 3.5-14.0 | 5.25-8.75 | 6.30-7.70 | 7.00 |
| Cod Liver Oil | 1.0-4.0 | 1.50-2.50 | 1.80-2.20 | 2.00 |
| Butylated | 0.25-1.0 | 0.375-0.625 | 0.45-0.55 | 0.50 |
| Hydroxy- | | | | |
| toluene | | | | |
| Propylparaben | 0.075-0.30 | 0.1125-0.1875 | 0.135-0.165 | 0.15 |
| Propylene | 9.0-27.0 | 13.5-22.5 | 16.2-19.8 | 18.00 |
| Glycol | | | | |
| Fragrance | 0.10-0.40 | 0.15-0.25 | 0.18-0.22 | 0.20 |

### Example 4

### Preparation of Aqueous Allantoin-Containing Composition in Gel Form with Polyalceryl Methacrylate Polymer

An aqueous anhydrous allantoin-containing composition in gel form with a polyglyceryl methacrylate polymer is prepared as shown in Table 4.

TABLE 4
AQUEOUS ALLANTOIN-CONTAINING COMPOSITION IN GEL
FORM WITH POLYGLYCERYL METHACRYLATE POLYMER

| | | | MORE | |
|---|---|---|---|---|
| | | | HIGHLY | OPTI- |
| INGREDIENT | RANGE | PREFERRED | PREFERRED | MUM |
| Allantoin | 0.5-10.0 | 1.125-1.875 | 1.35-1.65 | 1.50 |
| Water | 1.5-6.0 | 2.25-3.75 | 2.70-3.30 | 3.00 |
| Polyglyceryl | 55.0-85.0 | 60.0-80.0 | 66.0-74.0 | 67.65 |
| Methacrylate | | | | |
| Lanolin Oil | 3.5-14.0 | 5.25-8.75 | 6.30-7.70 | 7.00 |
| Cod Liver Oil | 1.0-4.0 | 1.50-2.50 | 1.80-2.20 | 2.00 |
| Butylated | 0.25-1.0 | 0.375-0.625 | 0.45-0.55 | 0.50 |
| Hydroxy- | | | | |
| toluene | | | | |
| Propylparaben | 0.075-0.30 | 0.1125-0.1875 | 0.135-0.165 | 0.15 |
| Fragrance | 0.10-0.40 | 0.15-0.25 | 0.18-0.22 | 0.20 |
| Propylene | 9.0-27.0 | 13.5-22.5 | 16.20-19.80 | 18.00 |
| Glycol | | | | |

### Example 5

### Preparation of Aqueous Allantoin-Containing Composition in Gel Form with Carboxypolymethylene Polymer and PEG-100 Stearate

An aqueous allantoin-containing composition in gel form with a carboxypolymethylene polymer and PEG-100 stearate is prepared as shown in Table 5. In Table 5, “Carbomer” refers to the carboxypolymethylene polymer and “Neolone” refers to the preservative 2-methyl-4-isothiazolin-3-one.

TABLE 5
AQUEOUS ALLANTOIN-CONTAINING
COMPOSITION IN GEL FORM WITH CARBOXY-
POLYMETHYLENE POLYMER AND PEG-100 STEARATE

| | | | MORE | |
|---|---|---|---|---|
| | | | HIGHLY | OPTI- |
| INGREDIENT | RANGE | PREFERRED | PREFERRED | MUM |
| Water | 60.0-90.0 | 65.0-88.0 | 75.0-85.0 | 78.47 |
| Allantoin | 0.5-10.0 | 1.125-1.875 | 1.35-1.65 | 1.50 |
| Carbomer | 0.5-2.0 | 0.75-1.25 | 0.90-1.10 | 1.00 |
| PEG-100 | 0.75-3.0 | 1.125-1.875 | 1.35-1.65 | 1.50 |
| Stearate | | | | |
| Lanolin Oil | 3.5-14.0 | 5.25-8.75 | 6.30-7.70 | 7.00 |
| Cod Liver Oil | 1.0-4.0 | 1.50-2.50 | 1.80-2.20 | 2.00 |
| NaOH | Sufficient to | Sufficient to | Sufficient to | Suffi- |
| | Adjust pH | Adjust pH | Adjust pH | cient to |
| | | | | Adjust |
| | | | | pH |
| Propylene | 4.0-16.0 | 6.0-10.0 | 7.20-8.80 | 8.00 |
| Glycol | | | | |
| Butylated | 0.25-1.0 | 0.375-0.625 | 0.45-0.55 | 0.50 |
| Hydroxy- | | | | |
| toluene | | | | |
| Neolone | 0.015-0.060 | 0.0225-0.0375 | 0.027-0.033 | 0.030 |

### Example 6

### Preparation of Aqueous Allantoin-Containing Composition in Gel Form Carboxypolymethylene Polymer and Complex with Glycerylpolyacrylate and Glycerin

An aqueous allantoin-containing composition in gel form with a carboxypolymethylene polymer and a complex with glycerylpolyacrylate and glycerin is prepared as shown in Table 6.

TABLE 6
AQUEOUS ALLANTOIN-CONTAINING COMPOSITION IN
GEL FORM WITH CARBOXYPOLYMETHYLENE POLYMER AND
COMPLEX WITH-GLYCERYLPOLYACRYLATE AND GLYCERIN

| | | | MORE | |
|---|---|---|---|---|
| | | | HIGHLY | OPTI- |
| INGREDIENT | RANGE | PREFERRED | PREFERRED | MUM |
| Water | 50.0-80.0 | 52.0-70.0 | 55.0-65.0 | 59.15 |
| Allantoin | 0.5-10.0 | 1.125-1.875 | 1.35-1.65 | 1.50 |
| Carbomer | 0.15-0.60 | 0.225-0.375 | 0.27-0.33 | 0.30 |
| Lanolin Oil | 3.5-14.0 | 5.25-8.75 | 6.30-7.70 | 7.00 |
| Cod Liver Oil | 1.0-4.0 | 1.50-2.50 | 1.80-2.20 | 2.00 |
| Glycerylpoly- | 10.0-30.0 | 15.0-25.0 | 18.0-22.0 | 20.00 |
| acrylate | | | | |
| /Glycerin | | | | |
| Triethanol- | 0.135-0.54 | 0.2025-0.3375 | 0.243-0.297 | 0.27 |
| amine | | | | |
| Propylene | 4.0-16.0 | 6.0-10.0 | 7.20-8.80 | 8.00 |
| Glycol | | | | |
| Sodium Lauro- | 0.5-2.0 | 0.75-1.25 | 0.90-1.10 | 1.00 |
| amphoacetate | | | | |
| Butylated | 0.25-1.0 | 0.375-0.625 | 0.45-0.55 | 0.50 |
| Hydroxy- | | | | |
| toluene | | | | |
| Fragrance | 0.10-0.40 | 0.15-0.25 | 0.18-0.22 | 0.20 |
| Propylparaben | 0.075-0.30 | 0.1125-0.1875 | 0.135-0.165 | 0.15 |
| Methylparaben | 0.10-0.40 | 0.15-0.25 | 0.18-0.22 | 0.20 |

### Example 7

### Preparation of Allantoin-Containing Composition for Administration as Aerosol by Mechanical Spray Pump

An allantoin-containing composition for administration as an aerosol by a mechanical spray pump is prepared as shown in Table 7.

TABLE 7
ALLANTOIN-CONTAINING COMPOSITION FOR
ADMINISTRATION AS AEROSOL BY MECHANICAL PUMP SPRAY

| | | | MORE- | |
|---|---|---|---|---|
| | | | HIGHLY | OPTI- |
| INGREDIENT | RANGE | PREFERRED | PREFERRED | MUM |
| Water | 50.0-90.0 | 60.0-80.0 | 65.0-75.0 | 72.735 |
| Allantoin | 0.50-10.0 | 1.0-2.0 | 1.35-1.65 | 1.50 |
| Sodium Tri- | 0.50-3.0 | 0.75-1.25 | 0.90-1.10 | 1.00 |
| deceth Sulfate | | | | |
| Lanolin Oil | 2.0-15.0 | 5.25-8.75 | 6.30-7.70 | 7.00 |
| Cod Liver Oil | 1.0-3.0 | 1.50-2.50 | 1.80-2.20 | 2.00 |
| Butylated | 0.10-1.0 | 0.375-0.625 | 0.45-0.55 | 0.50 |
| Hydroxy- | | | | |
| toluene | | | | |
| Fragrance | 0.05-0.50 | 0.15-0.25 | 0.18-0.22 | 0.20 |
| 2-Methyl-4- | 0.01-0.1 | 0.0225-0.0375 | 0.027-0.33 | 0.030 |
| isothiazolin- | | | | |
| 3-one | | | | |
| Propylene | 5.0-30.0 | 11.25-18.75 | 13.50-16.50 | 15.00 |
| Glycol | | | | |
| Citric Acid | 0.10-0.10 | 0.02-0.08 | 0.025-0.040 | 0.035 |

### Example 8

### Preparation of Allantoin-Containing Composition for Administration as Pressurized Aerosol

An allantoin-containing composition for administration as a pressurized aerosol by a mechanical spray pump is prepared as shown in Table 8.

TABLE 8
ALLANTOIN-CONTAINING COMPOSITION FOR
ADMINISTRATION AS PRESSURIZED AEROSOL

| | | | MORE | |
|---|---|---|---|---|
| INGRE- | | | HIGHLY | OPTI- |
| DIENT | RANGE | PREFERRED | PREFERRED | MUM |
| Allantoin | 0.50-10.0 | 1.0-2.0 | 1.35-1.65 | 1.50 |
| SD Alco- | 15.675-62.70 | 23.5125-39.1875 | 28.215-34.485 | 31.35 |
| hol 40 | | | | |
| Propylene | 7.50-30.0 | 11.25-18.75 | 13.50-16.50 | 15.00 |
| Glycol | | | | |
| Lanolin | 2.50-10.0 | 3.75-6.25 | 4.50-5.50 | 5.00 |
| Oil | | | | |
| Cod Liver | 1.0-4.0 | 1.50-2.50 | 1.80-2.20 | 2.00 |
| Oil | | | | |
| Propyl- | 0.075-0.30 | 0.1125-0.1875 | 0.135-0.165 | 0.15 |
| paraben | | | | |
| Propane | 11.25-45.0 | 16.875-28.125 | 20.25-24.75 | 22.50 |
| Butane | 11.25-45.0 | 16.875-28.125 | 20.25-24.75 | 22.50 |

### Example 9

### Preparation of Allantoin-Containing Composition for Administration as Pressurized Aerosol Without Alcohol

An allantoin-containing composition for administration as a pressurized aerosol by a mechanical spray pump without alcohol is prepared as shown in Table 9.

TABLE 9
ALLANTOIN-CONTAINING COMPOSITION FOR ADMIN-
ISTRATION AS PRESSURIZED AEROSOL WITHOUT ALCOHOL

| | | | MORE | |
|---|---|---|---|---|
| INGRE- | | | HIGHLY | OPTI- |
| DIENT | RANGE | PREFERRED | PREFERRED | MUM |
| Allantoin | 0.50-10.0 | 1.0-2.0 | 1.35-1.65 | 1.50 |
| Propylene | 23.175-92.70 | 34.7625-57.9375 | 41.715-50.985 | 46.35 |
| Glycol | | | | |
| Lanolin | 2.50-10.0 | 3.75-6.25 | 4.50-5.50 | 5.00 |
| Oil | | | | |
| Cod Liver | 1.0-4.0 | 1.50-2.50 | 1.80-2.20 | 2.00 |
| Oil | | | | |
| Propyl- | 0.075-0.30 | 0.1125-0.1875 | 0.135-0.165 | 0.15 |
| paraben | | | | |
| Propane | 11.25-45.0 | 16.875-28.125 | 20.25-24.75 | 22.50 |
| Butane | 11.25-45.0 | 16.875-28.125 | 20.25-24.75 | 22.50 |

The present invention provides allantoin-containing compositions that offer improved ease of application as gels and aerosols. These compositions are particularly suitable for use in treating a number of skin diseases or conditions that are characterized by ulceration, inflammation, or blistering of the skin. These skin conditions or diseases include, but are not limited to, epidermolysis bullosa, decubitus ulcers, pressure ulcers, diabetic ulcers, milia, burns, eczema, urticaria, atopic dermatitis, contact dermatitis, arthritis, gout, and lupus erythematosus. Compositions according to the present invention are particularly suitable for treatment of epidermolysis bullosa. Compositions according to the present invention are convenient to store, dispense, and administer, and are well tolerated by patients. They can be used together with other treatments.

While the specification describes particular embodiments of the present invention, those of ordinary skill can devise variations of the present invention without departing from the inventive concept.

1. An allantoin-containing composition comprising:
(a) allantoin in a therapeutically effective quantity;
(b) a complex comprising mineral oil and at least one hydrogenated alkylene copolymer;
(c) lanolin oil; and
(d) cod liver oil, wherein the composition forms a gel.

2. The composition of claim 1, further comprising water.

3. The composition of claim 1, wherein at least one monomer in the hydrogenated alkylene copolymer is styrene.

4. The composition of claim 3, wherein the hydrogenated alkylene copolymer is a hydrogenated butylene/ethylene/styrene copolymer and a hydrogenated ethylene/propylene/styrene copolymer.

5. The composition of claim 1, further comprising a solvent component.

6. The composition of claim 5, wherein the solvent component comprises at least one solvent selected from the group consisting of ethylene glycol, propylene glycol, butylene glycol, and glycerin.

7. The composition of claim 1, further comprising an antioxidant component.

8. The composition of claim 1, further comprising a preservative component.

9. An allantoin-containing composition, comprising:
(a) allantoin in a therapeutically effective quantity;
(b) a complex comprising:
(i) mineral oil;
(ii) a hydrogenated butylene/ethylene/styrene copolymer; and
(iii) a hydrogenated ethylene/propylene/styrene copolymer;
(c) lanolin oil;
(d) cod liver oil;
(e) butylated hydroxytoluene;
(f) propylparaben;
(g) fragrance;
(h) propylene glycol; and
(i) allantoin, wherein the composition forms a gel.

10. The composition of claim 9, further comprising water.

11. A method of treating a skin condition or disease characterized by ulceration, inflammation, or blistering of the skin, comprising the step of applying to the skin the allantoin-containing composition of claim 1 in a therapeutically effective quantity.

12. The method of claim 11, wherein the skin condition or disease is selected from the group consisting of epidermolysis bullosa, decubitus ulcers, pressure ulcers, diabetic ulcers, milia, burns, eczema, urticaria, atopic dermatitis, contact dermatitis, arthritis, gout, and lupus erythematosus.

13. The method of claim 11, further comprising the step of administering an additional therapeutic agent in a therapeutically effective quantity.

14. A method of treating a skin condition or disease characterized by ulceration, inflammation, or blistering of the skin comprising the step of applying to the skin the allantoin-containing composition of claim 9 in a therapeutically effective quantity.

15. The method of claim 14, wherein the skin condition or disease is selected from the group consisting of epidermolysis bullosa, decubitus ulcers, pressure ulcers, diabetic ulcers, milia, burns, eczema, urticaria, atopic dermatitis, contact dermatitis, arthritis, gout, and lupus erythematosus.

16. The method of claim 14, further comprising the step of administering an additional therapeutic agent in a therapeutically effective quantity.

17. An allantoin-containing composition, comprising:
(a) allantoin in a therapeutically effective quantity;
(b) a polymer selected from the group consisting of polyglyceryl methacrylate, polyglyceryl acrylate, and polyglyceryl ethacrylate;
(c) lanolin oil; and
(d) cod liver oil, wherein the composition forms a gel.

18. The composition of claim 17, further comprising water.

19. The composition of claim 17, further comprising a solvent component.

20. The composition of claim 19, wherein the solvent component comprises at least one solvent selected from the group consisting of ethylene glycol, propylene glycol, butylene glycol, and glycerin.

21. The composition of claim 17, further comprising an antioxidant component.

22. The composition of claim 17, further comprising a preservative component.

23. The composition of claim 17, further comprising a fragrance.

24. A method of treating a skin condition or disease characterized by ulceration, inflammation, or blistering of the skin, comprising the step of applying to the skin the allantoin-containing composition of claim 17 in a therapeutically effective quantity.

25. The method of claim 24, wherein the skin condition or disease is selected from the group consisting of epidermolysis bullosa, decubitus ulcers, pressure ulcers, diabetic ulcers, milia, burns, eczema, urticaria, atopic dermatitis, contact dermatitis, arthritis, gout, and lupus erythematosus.

26. The method of claim 24, further comprising the step of administering an additional therapeutic agent in a therapeutically effective quantity.

27. An allantoin-containing composition, comprising:
(a) water;
(b) allantoin in a therapeutically effective quantity;
(c) a carboxypolymethylene polymer;
(d) PEG-100 stearate;
(e) lanolin oil;
(f) cod liver oil; and
(g) a water-soluble base to adjust the pH from about 3.5 to about 6.0.

28. The composition of claim 27, wherein the water-soluble base is sodium hydroxide.

29. The composition of claim 27, wherein the carboxypolymethylene polymer is a cross-linked polyacrylic acid that is cross-linked by one of allylsucrose and allylpentaerythritol with the polyacrylic acid.

30. The composition of claim 27, further comprising a solvent component.

31. The composition of claim 30, wherein the solvent component comprises at least one solvent selected from the group consisting of ethylene glycol, propylene glycol, butylene glycol, and glycerin.

32. The composition of claim 27, further comprising an antioxidant component.

33. The composition of claim 27, further comprising a preservative component.

34. A method of treating a skin condition or disease characterized by ulceration, inflammation, or blistering of the skin comprising the step of applying to the skin the allantoin-containing composition of claim 34 in a therapeutically effective quantity.

35. The method of claim 34, wherein the skin condition or disease is selected from the group consisting of epidermolysis bullosa, decubitus ulcers, pressure ulcers, diabetic ulcers, milia, burns, eczema, urticaria, atopic dermatitis, contact dermatitis, gout, and lupus erythematosus.

36. The method of claim 34, further comprising the step of administering an additional therapeutic agent in a therapeutically effective quantity.

37. An allantoin-containing composition, comprising:
(a) water;
(b) allantoin in a therapeutically effective quantity;
(c) a carboxypolymethylene polymer;
(d) lanolin oil;
(e) cod liver oil;
(f) a complex comprising:
(f) a complex comprising:
(i) a polymer selected from the group consisting of glycerylpolyacrylate, glycerylpolymethacrylate, and glycerylpolyethacrylate; and
(ii) glycerin; and
(g) a weak organic base to adjust the pH, wherein the composition forms a gel.

38. The composition of claim 37, wherein the weak organic base is triethanolamine.

39. The composition of claim 37, wherein the carboxypolymethylene polymer is a cross-linked polyacrylic acid that is cross-linked by one of allylsucrose and ailylpentaerythritol with the polyacrylic acid.

40. The composition of claim 37, further comprising a solvent component.

41. The composition of claim 40, wherein the solvent component comprises at least one solvent selected from the group consisting of ethylene glycol, propylene glycol, butylene glycol, and glycerin.

42. The composition of claim 37, further comprising an antioxidant component.

43. The composition of claim 37, further comprising a preservative component.

44. The composition of claim 37, further comprising sodium lauroamphoacetate.

45. A method of treating a skin condition or disease characterized by ulceration, inflammation, or blistering of the skin comprising the step of applying to the skin the allantoin-containing composition of claim 37, in a therapeutically effective quantity.

46. The method of claim 45, wherein the skin condition or disease is selected from the group consisting of epidermolysis bullosa, decubitus ulcers, pressure ulcers, diabetic ulcers, burns, milia, eczema, urticaria, atopic dermatitis, contact dermatitis, gout, and lupus erythematosus.

47. The method of claim 45, further comprising the step of administering an additional therapeutic agent in a therapeutically effective quantity.

48. An allantoin-containing composition, comprising:
(a) water;
(b) allantoin in a therapeutically effective quantity;
(c) an anionic ethoxylated surfactant;
(d) lanolin oil;
(e) cod liver oil; and
(f) an acid to adjust the pH to a value in a range from about 3.5 to about 6.0,
the composition having a viscosity and surface tension in a range that allows the composition to be administered to the skin of a user as an aerosol by a mechanical spray pump.

49. The composition of claim 48, wherein the anionic ethoxylated surfactant is selected from the group consisting of sodium trideceth sulfate, sodium dodeceth sulfate, ammonium trideceth sulfate, ammonium dodeceth sulfate, ethoxylated sodium oleyl succinate, ethoxylated ammonium lauryl sulfosuccinate, ethoxylated sodium dodecylbenzenesulfonate, and ethoxylated sodium N-lauryl sarcosinate.

50. The composition of claim 48, wherein the acid is a water-soluble weak organic acid.

51. The composition of claim 48, wherein the acid is selected from the group consisting of citric acid, ascorbic acid, glycolic acid, lactic acid, benzoic acid, and salicylic acid.

52. The composition of claim 48, wherein the acid is an inorganic acid.

53. The composition of claim 48, further comprising a solvent component.

54. The composition of claim 53, wherein the solvent component comprises at least one solvent selected from the group consisting of ethylene glycol, propylene glycol, butylene glycol, and glycerin.

55. The composition of claim 48, further comprising an antioxidant component.

56. The composition of claim 48, further comprising a preservative component.

57. The composition of claim 48, further comprising a fragrance component.

58. A method of treating a skin condition or disease characterized by ulceration, inflammation, or blistering of the skin, comprising the step of applying to the skin the allantoin-containing composition of claim 48 in a therapeutically effective quantity.

59. The method of claim 58, wherein the skin condition or disease is selected from the group consisting of epidermolysis bullosa, decubitus ulcers, pressure ulcers, diabetic ulcers, milia, burns, eczema, urticaria, atopic dermatitis, contact dermatitis, arthritis, gout, and lupus erythematosus.

60. The method of claim 58, further comprising the step of administering an additional therapeutic agent in a therapeutically effective quantity.

61. An allantoin-containing composition, comprising:
(a) allantoin in a therapeutically effective quantity;
(b) a substantially anhydrous hydroxylic solvent in a quantity sufficient to dissolve the allantoin;
(c) at least one inert hydrocarbon propellant;
(d) lanolin oil; and
(e) cod liver oil,
the composition having a viscosity and surface tension in a range that allows the composition to be administered to the skin of a user as a pressurized aerosol.

62. The composition of claim 61, wherein the substantially anhydrous hydroxylic solvent is an alcohol.

63. The composition of claim 61, wherein the inert hydrocarbon propellant comprises at least one hydrocarbon selected from the group consisting of propane, butane, isobutane, pentane, isopentane, and neopentane.

64. The composition of claim 61, further comprising a cosolvent component.

65. The composition of claim 64, wherein the cosolvent component comprises at least one cosolvent selected from the group consisting of ethylene glycol, propylene glycol, butylene glycol, and glycerin.

66. The composition of claim 61, further comprising a preservative component.

67. A method of treating a condition or disease characterized by ulceration, inflammation, or blistering of the skin comprising the step of administering to the skin the allantoin-containing composition of claim 61 in a therapeutically effective quantity.

68. The method of claim 67, wherein the skin condition or disease is selected from the group consisting of epidermolysis bullosa, decubitus ulcers, pressure ulcers, diabetic ulcers, milia, burns, eczema, urticaria, atopic dermatitis, contact dermatitis, arthritis, gout, and lupus erythematosus.

69. The method of claim 67, further comprising the step of administering an additional therapeutic agent in a therapeutically effective quantity.

70. An allantoin-containing composition, comprising:
(a) allantoin in a therapeutically effective quantity;
(b) propylene glycol in a quantity sufficient to dissolve the allantoin;
(c) at least one inert hydrocarbon propellant;
(d) lanolin oil;
(e) cod liver oil; and
(f) the composition having a viscosity and surface tension in a range that allows the composition to be administered to the skin of a user as a pressurized aerosol.

71. The composition of claim 70, wherein the inert hydrocarbon propellant comprises at least one hydrocarbon selected from the group consisting of propane, butane, isobutane, pentane, isopentane, and neopentane.

72. The composition of claim 70, further comprising a preservative component.

73. A method of treating a condition or disease characterized by ulceration, inflammation, or blistering of the skin comprising the step of administering to the skin the allantoin-containing composition of claim 70 in a therapeutically effective quantity.

74. The method of claim 73, wherein the skin condition or disease is selected from the group consisting of epidermolysis bullosa, decubitus ulcers, pressure ulcers, diabetic ulcers, milia, burns eczema, urticaria, atopic dermatitis, contact dermatitis, arthritis, gout, and lupus erythematosus.

75. The method of claim 73, further comprising the step of administering an additional therapeutic agent in a therapeutically effective quantity.
